SEQUENCE LISTING (144 sequences)

SEQ ID NO: 1 - DNA, 23 bp, Artificial Sequence
  synthetic oligonucleotide
tggtctcatcagttattcccatc

SEQ ID NO: 2 - DNA, 21 bp, Artificial Sequence
  synthetic oligonucleotide
cgagaaatgatggagtctgga

SEQ ID NO: 3 - DNA, 21 bp, Artificial Sequence
  synthetic oligonucleotide
tgatcttcatgctcagatcca

SEQ ID NO: 4 - DNA, 19 bp, Artificial Sequence
  synthetic oligonucleotide
gcaaatgttgttggcaagc

SEQ ID NO: 5 - DNA, 22 bp, Artificial Sequence
  synthetic oligonucleotide
tgaataaaaaggcaacctaccg

SEQ ID NO: 6 - DNA, 22 bp, Artificial Sequence
  synthetic oligonucleotide
tgggagatgtcaatctctgttc

SEQ ID NO: 7 - DNA, 25 bp, Artificial Sequence
  synthetic oligonucleotide
tgagaaaagcttctaaacagaaagg

SEQ ID NO: 8 - DNA, 20 bp, Artificial Sequence
  synthetic oligonucleotide
gtactggccctggtttaacg

SEQ ID NO: 9 - DNA, 21 bp, Artificial Sequence
  synthetic oligonucleotide
aagcaaagtccacacttccag

SEQ ID NO: 10 - DNA, 20 bp, Artificial Sequence
  synthetic oligonucleotide
gtagtaccgggaggctgacc

SEQ ID NO: 11 - DNA, 19 bp, Artificial Sequence
  synthetic oligonucleotide
ggatgtggtggacatcgtg

SEQ ID NO: 12 - DNA, 18 bp, Artificial Sequence
  synthetic oligonucleotide
gaggccgatggttctcag

SEQ ID NO: 13 - DNA, 20 bp, Artificial Sequence
  synthetic oligonucleotide
tattccatcggcttggtttg

SEQ ID NO: 14 - DNA, 20 bp, Artificial Sequence
  synthetic oligonucleotide
tgtgagctgaagacacagca

SEQ ID NO: 15 - DNA, 19 bp, Artificial Sequence
  synthetic oligonucleotide
ggccagatgaccgtctacc

SEQ ID NO: 16 - DNA, 18 bp, Artificial Sequence
  synthetic oligonucleotide
gccgtctgcacagttggt

SEQ ID NO: 17 - DNA, 20 bp, Artificial Sequence
  synthetic oligonucleotide
tctaccatgagcatcggtgt

SEQ ID NO: 18 - DNA, 21 bp, Artificial Sequence
  synthetic oligonucleotide
catcgaagacctcagagccta

SEQ ID NO: 19 - DNA, 18 bp, Artificial Sequence
  synthetic oligonucleotide
agggattccggacctttg

SEQ ID NO: 20 - DNA, 20 bp, Artificial Sequence
  synthetic oligonucleotide
gcagcagtttgggttgtttc

SEQ ID NO: 21 - DNA, 20 bp, Artificial Sequence
  synthetic oligonucleotide
gccatcactgtgtcatctgc

SEQ ID NO: 22 - DNA, 20 bp, Artificial Sequence
  synthetic oligonucleotide
catcttgaatccaccgaagg

SEQ ID NO: 23 - DNA, 19 bp, Artificial Sequence
  synthetic oligonucleotide
catggtatcctccgcaatg

SEQ ID NO: 24 - DNA, 21 bp, Artificial Sequence
  synthetic oligonucleotide
gcgcatttaaggcattgtaac

SEQ ID NO: 25 - DNA, 21 bp, Artificial Sequence
  synthetic oligonucleotide
aacctggacaaagcactgaag

SEQ ID NO: 26 - DNA, 22 bp, Artificial Sequence
  synthetic oligonucleotide
aatgcattgtagcagttgatcc

SEQ ID NO: 27 - DNA, 21 bp, Artificial Sequence
  synthetic oligonucleotide
caagctggctgacttaatgga

SEQ ID NO: 28 - DNA, 20 bp, Artificial Sequence
  synthetic oligonucleotide
gactttcccagcattcatcg

SEQ ID NO: 29 - DNA, 21 bp, Artificial Sequence
  synthetic oligonucleotide
gaccggagaacgctgatacta

SEQ ID NO: 30 - DNA, 18 bp, Artificial Sequence
  synthetic oligonucleotide
agtcgggcagtcagcatc

SEQ ID NO: 31 - DNA, 20 bp, Artificial Sequence
  synthetic oligonucleotide
tccctacttcccgtctttga

SEQ ID NO: 32 - DNA, 19 bp, Artificial Sequence
  synthetic oligonucleotide
acaggctctgcccgattac

SEQ ID NO: 33 - DNA, 20 bp, Artificial Sequence
  synthetic oligonucleotide
cgctcgctcctctacatcat

SEQ ID NO: 34 - DNA, 21 bp, Artificial Sequence
  synthetic oligonucleotide
gccaacttcagcttgtttttg

SEQ ID NO: 35 - DNA, 20 bp, Artificial Sequence
  synthetic oligonucleotide
tgttcggggacgtaaaagac

SEQ ID NO: 36 - DNA, 20 bp, Artificial Sequence
  synthetic oligonucleotide
tgaggatttgcatcactggt

SEQ ID NO: 37 - DNA, 23 bp, Artificial Sequence
  synthetic oligonucleotide
tgtggagaagctcaaaaagtcac

SEQ ID NO: 38 - DNA, 20 bp, Artificial Sequence
  synthetic oligonucleotide
cccatagtcatgggactgct

SEQ ID NO: 39 - DNA, 21 bp, Artificial Sequence
  synthetic oligonucleotide
gctgaagcagttcaacaaagg

SEQ ID NO: 40 - DNA, 21 bp, Artificial Sequence
  synthetic oligonucleotide
aggagaggcaacaaggaagtc

SEQ ID NO: 41 - DNA, 20 bp, Artificial Sequence
  synthetic oligonucleotide
ctggatgaagccatcgagtt

SEQ ID NO: 42 - DNA, 22 bp, Artificial Sequence
  synthetic oligonucleotide
ccaacacctgtttgataacctg

SEQ ID NO: 43 - DNA, 20 bp, Artificial Sequence
  synthetic oligonucleotide
aagagttgctgcacatctgg

SEQ ID NO: 44 - DNA, 18 bp, Artificial Sequence
  synthetic oligonucleotide
gtgcagtgtggcctcaga

SEQ ID NO: 45 - DNA, 18 bp, Artificial Sequence
  synthetic oligonucleotide
accggttccgtaccttcc

SEQ ID NO: 46 - DNA, 21 bp, Artificial Sequence
  synthetic oligonucleotide
agctgtgcacaaaatggaaat

SEQ ID NO: 47 - DNA, 20 bp, Artificial Sequence
  synthetic oligonucleotide
aacagctggaaaggggtctc

SEQ ID NO: 48 - DNA, 25 bp, Artificial Sequence
  synthetic oligonucleotide
cattaagtcgtaccatttacggagt

SEQ ID NO: 49 - DNA, 21 bp, Artificial Sequence
  synthetic oligonucleotide
ggtaaatgccacatggtatcc

SEQ ID NO: 50 - DNA, 24 bp, Artificial Sequence
  synthetic oligonucleotide
tgattcaacaaattttccatcaat

SEQ ID NO: 51 - DNA, 20 bp, Artificial Sequence
  synthetic oligonucleotide
tgtccttcactgggagcact

SEQ ID NO: 52 - DNA, 20 bp, Artificial Sequence
  synthetic oligonucleotide
gtttcctccaagctccaaca

SEQ ID NO: 53 - DNA, 22 bp, Artificial Sequence
  synthetic oligonucleotide
ggcaagaatcctgctatcatct

SEQ ID NO: 54 - DNA, 22 bp, Artificial Sequence
  synthetic oligonucleotide
actggtacaaaggcaaatctcc

SEQ ID NO: 55 - DNA, 20 bp, Artificial Sequence
  synthetic oligonucleotide
ctggcagtgcctggaatact

SEQ ID NO: 56 - DNA, 19 bp, Artificial Sequence
  synthetic oligonucleotide
cctcctgggagctgaatgt

SEQ ID NO: 57 - DNA, 18 bp, Artificial Sequence
  synthetic oligonucleotide
ctcttgggccaccatgtc

SEQ ID NO: 58 - DNA, 19 bp, Artificial Sequence
  synthetic oligonucleotide
ctggccaggttttctcctg

SEQ ID NO: 59 - DNA, 22 bp, Artificial Sequence
  synthetic oligonucleotide
gatgtcatcgtcacagagaacg

SEQ ID NO: 60 - DNA, 18 bp, Artificial Sequence
  synthetic oligonucleotide
aagacacaggcgctgtcc

SEQ ID NO: 61 - DNA, 19 bp, Artificial Sequence
  synthetic oligonucleotide
gcctcaccaaacagattgc

SEQ ID NO: 62 - DNA, 18 bp, Artificial Sequence
  synthetic oligonucleotide
cacgcctcgcattaccat

SEQ ID NO: 63 - DNA, 20 bp, Artificial Sequence
  synthetic oligonucleotide
ggcgttcgtgattactgtga

SEQ ID NO: 64 - DNA, 21 bp, Artificial Sequence
  synthetic oligonucleotide
tcttcctcatgtccaaatcca

SEQ ID NO: 65 - DNA, 18 bp, Artificial Sequence
  synthetic oligonucleotide
ctttgccacggacgagac

SEQ ID NO: 66 - DNA, 22 bp, Artificial Sequence
  synthetic oligonucleotide
tcattgtactctgagggctgac

SEQ ID NO: 67 - DNA, 20 bp, Artificial Sequence
  synthetic oligonucleotide
caacacaaggaactgcttcg

SEQ ID NO: 68 - DNA, 20 bp, Artificial Sequence
  synthetic oligonucleotide
gatttagcatggggctcttg

SEQ ID NO: 69 - DNA, 21 bp, Artificial Sequence
  synthetic oligonucleotide
cataaggagttccttcgtctc

SEQ ID NO: 70 - DNA, 25 bp, Artificial Sequence
  synthetic oligonucleotide
cgactttcacgagtaacatcttctt

SEQ ID NO: 71 - DNA, 19 bp, Artificial Sequence
  synthetic oligonucleotide
gggaagccgcactgttact

SEQ ID NO: 72 - DNA, 19 bp, Artificial Sequence
  synthetic oligonucleotide
gagatcgcgcactgtttgt

SEQ ID NO: 73 - DNA, 20 bp, Artificial Sequence
  synthetic oligonucleotide
ctgatgctagtgctgccaga

SEQ ID NO: 74 - DNA, 20 bp, Artificial Sequence
  synthetic oligonucleotide
gtgcactctgaggccaaact

SEQ ID NO: 75 - DNA, 20 bp, Artificial Sequence
  synthetic oligonucleotide
gcccactaaaattcggaagc

SEQ ID NO: 76 - DNA, 22 bp, Artificial Sequence
  synthetic oligonucleotide
cttctgctacaaccactgaacg

SEQ ID NO: 77 - DNA, 20 bp, Artificial Sequence
  synthetic oligonucleotide
acaaaaagaggatgggcttg

SEQ ID NO: 78 - DNA, 20 bp, Artificial Sequence
  synthetic oligonucleotide
tcatgacggagcctttctct

SEQ ID NO: 79 - DNA, 20 bp, Artificial Sequence
  synthetic oligonucleotide
cttgggaaggcaagaaggta

SEQ ID NO: 80 - DNA, 20 bp, Artificial Sequence
  synthetic oligonucleotide
tggagctgctggtgttttct

SEQ ID NO: 81 - DNA, 19 bp, Artificial Sequence
  synthetic oligonucleotide
aaagcctcccccagaagtt

SEQ ID NO: 82 - DNA, 21 bp, Artificial Sequence
  synthetic oligonucleotide
catgttcaaattggcatgtgt

SEQ ID NO: 83 - DNA, 27 bp, Artificial Sequence
  synthetic oligonucleotide
caagtgtctttctggattaataaggag

SEQ ID NO: 84 - DNA, 24 bp, Artificial Sequence
  synthetic oligonucleotide
gtcatggttaaaacagtggtgatt

SEQ ID NO: 85 - DNA, 20 bp, Artificial Sequence
  synthetic oligonucleotide
ccctctggatgagcaaaact

SEQ ID NO: 86 - DNA, 21 bp, Artificial Sequence
  synthetic oligonucleotide
aattcgatgtcatccgtggta

SEQ ID NO: 87 - DNA, 25 bp, Artificial Sequence
  synthetic oligonucleotide
gggtctccttttggattaactatga

SEQ ID NO: 88 - DNA, 26 bp, Artificial Sequence
  synthetic oligonucleotide
ggtcattgttaggacagttgtaattc

SEQ ID NO: 89 - DNA, 21 bp, Artificial Sequence
  synthetic oligonucleotide
ctccattgtagagcaccgtct

SEQ ID NO: 90 - DNA, 22 bp, Artificial Sequence
  synthetic oligonucleotide
tcaatgaaagtcgaggataggc

SEQ ID NO: 91 - DNA, 21 bp, Artificial Sequence
  synthetic oligonucleotide
cggagctgatgaacttcaaat

SEQ ID NO: 92 - DNA, 18 bp, Artificial Sequence
  synthetic oligonucleotide
atgtagacgccccggttc

SEQ ID NO: 93 - DNA, 20 bp, Artificial Sequence
  synthetic oligonucleotide
acagaaaatgacgctgtgga

SEQ ID NO: 94 - DNA, 22 bp, Artificial Sequence
  synthetic oligonucleotide
catctgacttttggcttgtgaa

SEQ ID NO: 95 - DNA, 19 bp, Artificial Sequence
  synthetic oligonucleotide
atgcgacaccagcaagaac

SEQ ID NO: 96 - DNA, 20 bp, Artificial Sequence
  synthetic oligonucleotide
caatggtgctgagtgtggtc

SEQ ID NO: 97 - DNA, 20 bp, Artificial Sequence
  synthetic oligonucleotide
atttcaccctggtcaccgta

SEQ ID NO: 98 - DNA, 21 bp, Artificial Sequence
  synthetic oligonucleotide
gctcaaattgcaaaaccaatc

SEQ ID NO: 99 - DNA, 20 bp, Artificial Sequence
  synthetic oligonucleotide
tgcctgctagagtcccctta

SEQ ID NO: 100 - DNA, 19 bp, Artificial Sequence
  synthetic oligonucleotide
ccgtgatgatggtggacat

SEQ ID NO: 101 - DNA, 22 bp, Artificial Sequence
  synthetic oligonucleotide
gacattgatgtctccattctgc

SEQ ID NO: 102 - DNA, 21 bp, Artificial Sequence
  synthetic oligonucleotide
gcagccctttgtaaccataga

SEQ ID NO: 103 - DNA, 20 bp, Artificial Sequence
  synthetic oligonucleotide
gaacatggcagcgaaagtct

SEQ ID NO: 104 - DNA, 20 bp, Artificial Sequence
  synthetic oligonucleotide
ctggtacttgctgccaaaca

SEQ ID NO: 105 - DNA, 20 bp, Artificial Sequence
  synthetic oligonucleotide
agggatcgacatccagagag

SEQ ID NO: 106 - DNA, 20 bp, Artificial Sequence
  synthetic oligonucleotide
tgcacatttcctgtcaaacc

SEQ ID NO: 107 - DNA, 18 bp, Artificial Sequence
  synthetic oligonucleotide
ggggaggtgattccaagg

SEQ ID NO: 108 - DNA, 19 bp, Artificial Sequence
  synthetic oligonucleotide
cccccgacaaggatgtaga

SEQ ID NO: 109 - DNA, 20 bp, Artificial Sequence
  synthetic oligonucleotide
agccgtgtgatacgatgaaa

SEQ ID NO: 110 - DNA, 20 bp, Artificial Sequence
  synthetic oligonucleotide
caaggtttacaggcgttcct

SEQ ID NO: 111 - DNA, 20 bp, Artificial Sequence
  synthetic oligonucleotide
ctgccaacagttttgctgtg

SEQ ID NO: 112 - DNA, 20 bp, Artificial Sequence
  synthetic oligonucleotide
aaatggcaaacacccctcta

SEQ ID NO: 113 - DNA, 21 bp, Artificial Sequence
  synthetic oligonucleotide
agacttttggcaaagacatgc

SEQ ID NO: 114 - DNA, 19 bp, Artificial Sequence
  synthetic oligonucleotide
actgccattcaagcccttc

SEQ ID NO: 115 - DNA, 21 bp, Artificial Sequence
  synthetic oligonucleotide
ccctaccgtgatgtcttttca

SEQ ID NO: 116 - DNA, 20 bp, Artificial Sequence
  synthetic oligonucleotide
agaatgtccatgtcgccact

SEQ ID NO: 117 - DNA, 18 bp, Artificial Sequence
  synthetic oligonucleotide
tctggtttggcgttggag

SEQ ID NO: 118 - DNA, 22 bp, Artificial Sequence
  synthetic oligonucleotide
tcctccaactattctggtcgat

SEQ ID NO: 119 - DNA, 20 bp, Artificial Sequence
  synthetic oligonucleotide
agtgccaccataccatccag

SEQ ID NO: 120 - DNA, 21 bp, Artificial Sequence
  synthetic oligonucleotide
gctggcacttcagagacattc

SEQ ID NO: 121 - DNA, 22 bp, Artificial Sequence
  synthetic oligonucleotide
cacttcatcttcaccactctgg

SEQ ID NO: 122 - DNA, 19 bp, Artificial Sequence
  synthetic oligonucleotide
actcccgagtagcggtagg

SEQ ID NO: 123 - DNA, 20 bp, Artificial Sequence
  synthetic oligonucleotide
gccattgagtatggcacgat

SEQ ID NO: 124 - DNA, 20 bp, Artificial Sequence
  synthetic oligonucleotide
tggtaacgggaattttggaa

SEQ ID NO: 125 - DNA, 20 bp, Artificial Sequence
  synthetic oligonucleotide
cccctcagctagcctcatct

SEQ ID NO: 126 - DNA, 20 bp, Artificial Sequence
  synthetic oligonucleotide
gcctcgcaccagttcttcta

SEQ ID NO: 127 - DNA, 21 bp, Artificial Sequence
  synthetic oligonucleotide
catttagggctatcacctcca

SEQ ID NO: 128 - DNA, 22 bp, Artificial Sequence
  synthetic oligonucleotide
cactgtgtctttttggttttgc

SEQ ID NO: 129 - DNA, 19 bp, Artificial Sequence
  synthetic oligonucleotide
attcaaccagaggggcgta

SEQ ID NO: 130 - DNA, 21 bp, Artificial Sequence
  synthetic oligonucleotide
ttcaagacagctgcgtcatag

SEQ ID NO: 131 - DNA, 19 bp, Artificial Sequence
  synthetic oligonucleotide
gggttacaaccggtgccta

SEQ ID NO: 132 - DNA, 20 bp, Artificial Sequence
  synthetic oligonucleotide
ctttgccgatggtgaaagat

SEQ ID NO: 133 - DNA, 18 bp, Artificial Sequence
  synthetic oligonucleotide
gaagcgggccatagacct

SEQ ID NO: 134 - DNA, 18 bp, Artificial Sequence
  synthetic oligonucleotide
tggcagatccctgagagc

SEQ ID NO: 135 - DNA, 19 bp, Artificial Sequence
  synthetic oligonucleotide
tgcgatacaaccagccaag

SEQ ID NO: 136 - DNA, 20 bp, Artificial Sequence
  synthetic oligonucleotide
agatgaaggcgtccagtttc

SEQ ID NO: 137 - DNA, 22 bp, Artificial Sequence
  synthetic oligonucleotide
cacgaatcaaaaacaaatccaa

SEQ ID NO: 138 - DNA, 23 bp, Artificial Sequence
  synthetic oligonucleotide
tgtgtttaatgctctgtgaaacc

SEQ ID NO: 139 - DNA, 18 bp, Artificial Sequence
  synthetic oligonucleotide
cgtcctacggagggaggt

SEQ ID NO: 140 - DNA, 18 bp, Artificial Sequence
  synthetic oligonucleotide
ccagggacaccagcacat

SEQ ID NO: 141 - DNA, 20 bp, Artificial Sequence
  synthetic oligonucleotide
ctcaaggaggctacccacag

SEQ ID NO: 142 - DNA, 20 bp, Artificial Sequence
  synthetic oligonucleotide
tagttcccatgctgaggtga

SEQ ID NO: 143 - DNA, 20 bp, Artificial Sequence
  synthetic oligonucleotide
tctgtgcgagaggtagcaga

SEQ ID NO: 144 - DNA, 19 bp, Artificial Sequence
  synthetic oligonucleotide
cggatgaagatggcgatag

(19) United States
(12) Patent Application Publication
(10) Pub. No.: US 2018/0008620 A1
(43) Pub. Date: Jan. 11, 2018

(54) METHODS OF TREATING NEUROLOGICAL INFLAMMATORY DISORDERS

(71) Applicant: Mayo Foundation for Medical Education and Research, Rochester, MN (US)

(72) Inventor: Charles L. Howe, Rochester, MN (US)

(73) Assignee: Mayo Foundation for Medical Education and Research, Rochester, MN (US)

(21) Appl. No.: 15/543,838

(22) PCT Filed: Jan. 15, 2016
(86) PCT No.: PCT/US2016/013622

§ 371 Date: Jul. 14, 2017

Related U.S. Application Data
(60) Provisional application No. 62/103,629, filed on Jan. 15, 2015.

(51) Int. Cl. A61K 31/675

(52) U.S. Cl. CPC A61K 31/675

(57) ABSTRACT

Methods of treating neurological inflammatory disease or seizures caused by neuroinflammation by administering cPMP are described. Treating neuroinflammatory and neurometabolic diseases with cPMP is described so as to override dyshomeostasis in the MoCo synthesis pathway and control synaptic inhibition in the gephyrin-GABAR pathway. This is a novel strategy for preventing neural circuit dyshomeostasis by stabilizing inhibitory synapses.

### CROSS REFERENCE TO RELATED APPLICATIONS

This application claims the benefit of U.S. Provisional Ser. No. 62/103,629 filed Jan. 15, 2015. This disclosure of the prior application is considered part of (and is incorporated by reference in) the disclosure of this application.

### TECHNICAL FIELD

This disclosure generally relates to methods of treating neurological inflammatory diseases.

### BACKGROUND

Molybdenum cofactor (MoCo) is an evolutionarily conserved molybedenum (Mo) coordinated pterin-compound and is necessary for the activity of all Mo-enzymes, with the exception of nitrogenase. MoCo is synthesized by a unique and evolutionarily conserved multi-step pathway, from which only two intermediates have been identified: the sulphur- and metal-free pterin derivative, precursor Z, also known as cPMP, and molybdopterin (MPT), a pterin with an ene-dithiol function, which is essential for the Mo-linkage.

### SUMMARY

Methods of treating neurological inflammatory disease or seizures caused by neuroinflammation by administering cPMP are described. Treating neuroinflammatory and neurometabolic diseases with cPMP is described so as to override dyshomeostasis in the MoCo synthesis pathway and control synaptic inhibition in the gephyrin-GABAR pathway. This is a novel strategy for preventing neural circuit dyshomeostasis by stabilizing inhibitory synapses.

In one aspect, a method of treating a neurological inflammatory disease in an individual is provided. Such a method typically includes administering an effective amount of cPMP to the individual, thereby treating the individual. Representative neurological inflammatory diseases include, without limitation, central nervous system (CNS) autoimmune disorders such as multiple sclerosis (MS), neuromyelitis optica (NMO), anti-NMDA receptor encephalitis, and autoimmune epilepsies; Alzheimer's disease; amyotrophic lateral sclerosis (ALS); schizophrenia; autism; epilepsy and other seizure disorders (e.g., febrile seizures without underlying infection); CNS infectious diseases (e.g., viral, bacterial, parasitic); MoCo deficiencies; and other neurodegenerative diseases involving microglial and astrocytic inflammatory responses.

In some embodiments, the administering step is selected from the group consisting of orally, topically, and parenterally. In some embodiments, such a method further includes identifying an individual having a neurological inflammatory disease (e.g., identifying an individual having ALS, epilepsy or another seizure disorder, or autism or schizophrenia). In some embodiments, such a method further includes identifying an individual having a mutation in a gene selected from the group consisting of gephyrin, MOCS1, and MOCS2. In some embodiments, such a method further includes monitoring the individual for the amount of MPT, MoCo, MoCo—S or another intermediate or by-product of the MoCo biosynthesis pathway.

In another aspect, a method of treating ALS, epilepsy or another seizure disorder in an individual is provided. Such a method generally includes administering an effective amount of cPMP to the individual, thereby treating the individual. In some embodiments, the administering step is selected from the group consisting of orally, topically, and parenterally. In some embodiments, such a method further includes identifying an individual having ALS, epilepsy or another seizure disorder. In some embodiments, such a method further includes monitoring the individual for the amount of MPT, MoCo, MoCo—S or another intermediate or by-product of the MoCo biosynthesis pathway.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1A is a schematic showing the synthesis of molybdenum co-factor under homeostatic conditions.

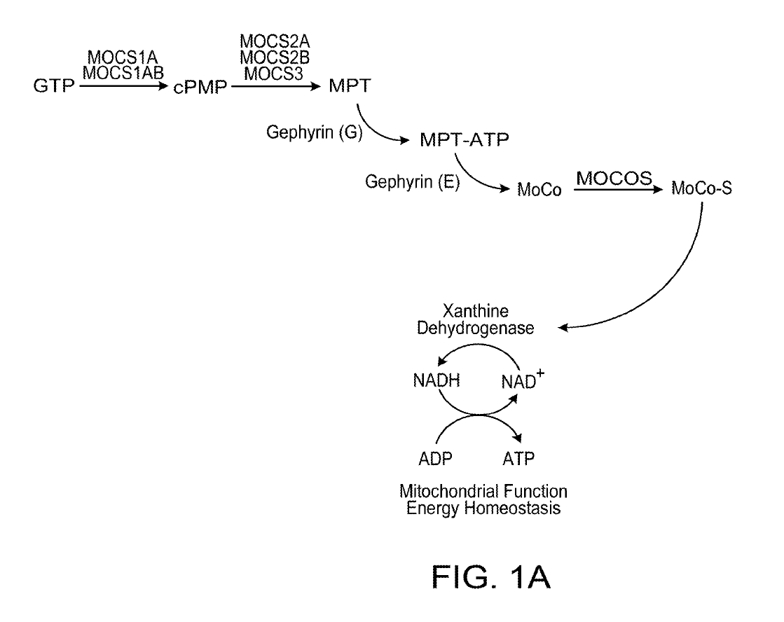

FIG. 1B are the chemical structures of the first three compounds shown in FIG. 1A: guanosine triphosphate (GTP), cyclic pyranopterin monophosphate (cPMP), and molybdopterin (MPT).

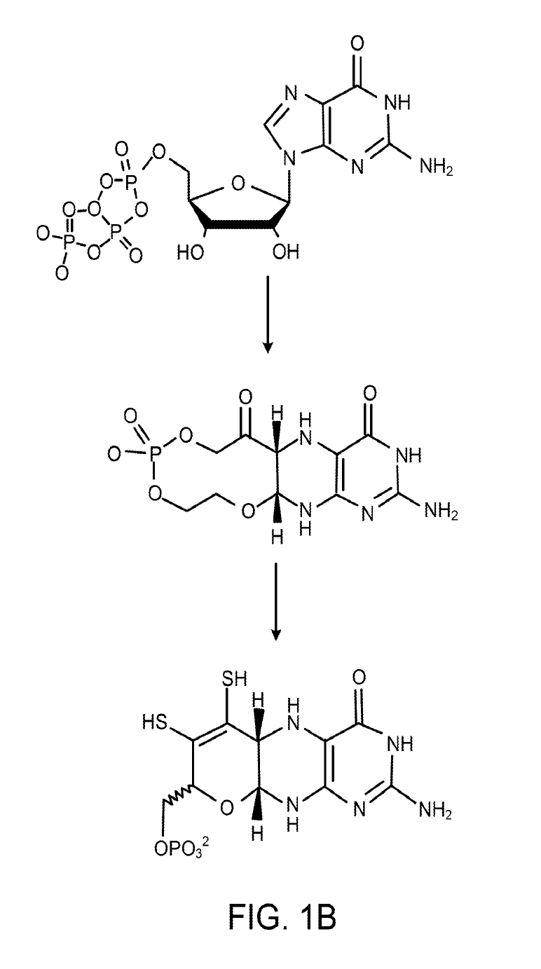

FIG. 2 is a schematic showing the synthesis of molybdenum co-factor during inflammatory conditions.

FIG. 3 is a schematic showing a summary of the impact of inflammatory cytokines on elements of the molybdenum biosynthesis pathway and the concomitant dysregulation of inhibitory synaptic function that results in hyperexcitability and seizure.

FIG. 4 demonstrates the IFNgamma-induced dysregulation of the MoCo pathway and down-regulation of inhibitory synaptic proteins. Mouse cortical neurons were cultured in a two-chamber device that separates cell bodies from axons. Panel A is a schematic of the chambered device constructed in PDMS polymer. Panel B is a low-magnification image of the corresponding regions shown in Panel A stained with an antibody against neurofilament (an axon-specific protein). Higher magnification images are shown of the cell body chamber (Panel C), the axon grooves (Panel D), and the axon chamber (Panel E). DAPI staining indicates the complete absence of any cells in the axon chamber. Panel F is a schematic showing the experimental design: IFNgamma was added to the pure axons in the axon chamber; RNA was collected from the cell body chamber 72 hours later and analyzed by microarray to identify changes in gene expression.

FIG. 5 are graphs showing that IFNgamma stimulation of the distal axons stimulated a transcriptional program in the neuron cell bodies that is marked by simultaneous down-regulation of numerous components of inhibitory synapses (gephyrin (Panel A), glycine receptor beta subunit (Panel B), numerous GABA receptor elements (not shown), and multiple gephyrin-binding scaffolds (not shown)) and robust up-regulation of MOCOS (Panel C). At the same time, GTP cyclohydrolase I (Panel D), xanthine dehydrogenase (Panel E), and aldehyde dehydrogenase (Panel F) were significantly up-regulated, indicating substantial changes in the MoCo pathway.

FIG. 6 shows spontaneous calcium levels in neurons stimulated for 24 hr with TNF-alpha or unstimulated (PBS, vehicle control). The PBS control cultures show low amplitude calcium transients that are non-synchronous. In contrast, stimulation with TNFalpha drove the cells to exhibit large amplitude calcium signals that were highly synchronized, indicating a general reduction in synaptic inhibition in the neural network. Calcium levels were monitored using a fluorescent reporter transduced into the neurons with adenovirus.

FIG. 7 demonstrates that spontaneous activity in neurons stimulated with TNFalpha or IFNgamma is highly correlated (hence, synchronous), in contrast to vehicle control-stimulated cultures. The Pearson correlation matrix was calculated for all of the neurons showing spontaneous activity in the culture and heat-mapped (red=highly correlated activity; blue=uncorrelated activity). In the vehicle control neurons, there is a small cluster of synchronous activity but the overall network is uncorrelated. TNFalpha or IFNgamma stimulation resulted in nearly complete correlation across the entire network. Moreover, the absolute number of spontaneously active cells is clearly increased in the IFNgamma and TNFalpha stimulated networks (137 neurons and 151 neurons, respectively, versus only 42 neurons in the vehicle control). These findings indicate that inhibition in the network is highly suppressed by TNFalpha or IFNgamma stimulation.

FIG. 8 are graphs showing the averaged calcium responses in cytokine-stimulated neurons. FIG. 8A shows the basal levels of activity in the neuron cultures. Non-synchronized calcium responses occur in the control cultures, resulting in an overall low level of synaptic activity in the network. FIGS. 8B and 8C show that stimulation with IFN gamma or TNF alpha results in network bursting and highly synchronized synaptic activity in which many cells in the culture flux calcium at the same time. FIG. 8D shows that treatment of control cultures with picrotoxin (2.4 μM), a small molecule inhibitor of inhibitory GABAergic channels, induces network synchrony and bursting that phenocopies the response observed in cytokine-stimulated cultures. FIG. 8E shows that the addition of GABA (27 μM) to control cultures completely suppresses synaptic activity, consistent with enhanced inhibition.

### DETAILED DESCRIPTION

As described herein, modulation of the molybdenum cofactor biosynthetic pathway returns cells that are under inflammatory stress to homeostasis by reducing shunting of gephyrin away from stabilization of inhibitory synapses and renormalizing inhibitory control of neural networks. Therefore, as described herein, cPMP can be administered to an individual to treat a number of different neurological inflammatory diseases or relieve the symptoms that are a result of a number of different neurological disorders.

### Molybdenum Cofactor (MoCo) and the Genetics Associated Therewith

All of the molybdenum (Mo) containing enzymes of humans, animals, plants, arachaea and bacteria, with the exception of nitrogenase from prokaryotes, require a co-factor that includes an organic moiety, molybdopterin (MPT), and molybdenum. This molybdenum cofactor (MoCo) possesses, across all phylogenetic groups, the same base structure that is very unstable in its free form, in particular under aerobic conditions when it is not bound to an apoprotein. The biosynthetic pathway, discussed in more detail below, is evolutionarily conserved and the corresponding proteins from various organisms are extremely homologous.

A mutational defect in MoCo-biosynthesis leads to simultaneous loss of the activities of all Mo enzymes, inclusive the sulphite oxidase. Human MoCo deficiency is a severe, autosomal-recessive genetic disorder, which clinically cannot be differentiated from the less frequently occurring sulphite-oxidase deficiency. Most afflicted patients exhibit neurological abnormalities such as non-treatable seizures and lack of development of the brain, which can be traced back to the toxicity of sulphite, a lack of sulphate or both. Most afflicted patients usually die in early childhood.

A eukaryotic gene encoding a protein involved in MoCo-biosynthesis was obtained from Arabidopsis thaliana. Subsequently, a human gene encoding a protein involved in MoCo-biosynthesis, was obtained and designated MOCS1. Due to alternate splicing of the MOCS1 gene, the MOCS1A and MOCS1B proteins are produced and convert a guanosine derivative into the sulphur-free precursor Z (i.e., cPMP). Patients having a mutation in the MOCS1 gene are referred to as having MoCo-deficiency type A. In a subsequent step, precursor Z (i.e., cPMP) is converted to MPT by an enzyme, which is encoded by a gene designated MOCS2 and activated by the protein encoded by the MOCS3 gene. Patients having a mutation in the MOCS2 gene are referred to as having MoCo-deficiency type B. Finally, Mo is inserted into MPT by a protein referred to as gephyrin. Patients having a mutation in the gene encoding gephyrin, GEPHN, are referred to as having MoCo-deficiency type C.

### Inflammation-Induced Injury and the MoCo Biosynthesis Pathway

Inflammation triggers neuronal and axonal injury via multiple mechanisms. However, perhaps the most physiologically relevant mechanism of neuronal injury is inflammation-induced synaptic dysfunction and derailment of homeostatic electrophysiological activity in neural circuits. For example, TNFalpha and IFNgamma are known to induce hippocampal injury by triggering excitotoxicity. There are multiple mechanism by which these inflammatory cytokines alter synaptic function—for example, by altering excitatory receptor function and increasing synaptic calcium levels. However, an equally important mechanism of cytokine-mediated synaptic dysregulation may be down-regulation of inhibitory receptors. Reduced inhibition will raise the overall level of synaptic activity and create a feedback loop in which excitatory synaptic activity builds, calcium accumulates in the synapse, and calcium-dependent proteases degrade synaptic connections. This feedback loop likely exacerbates the loss of inhibition, creating spreading synaptic dysregulation, neural injury, and neural circuit hyperactivity and/or failure.

Gephyrin is a critical scaffolding protein that controls the localization, clustering, and inhibitory function of glycine and GABA receptors at synaptic sites. Gephyrin function is directly tied to inhibitory control of neural circuitry, and down-regulation of gephyrin is linked to seizures and hyperexcitability of neurons. Genetic defects in gephyrin are associated with autism, epilepsy, and schizophrenia.

The inhibitory receptor scaffolding function of gephyrin is mediated by a C domain that links evolutionarily conserved G and E domains. Crucially, the G and E domains of gephyrin are necessary for the synthesis of molybdenum cofactor (MoCo), a molecule that is required for activation of molybdenum-dependent enzymes necessary for survival. Humans with mutations in the non-scaffolding domains of gephyrin exhibit MoCo deficiencies and severe neurological and developmental abnormalities.

Under homeostatic conditions, guanosine triphosphate (GTP) is converted to a coordination complex of molybdopterin and a molybdenum oxide (MoCo) by the action of several catalytic enzymes including gephyrin. MoCo must be sulfurated by the molybdenum cofactor sulfurase (MOCOS) in order to function as a co-factor for xanthine dehydrogenase and other molybdenum-dependent enzymes. Xanthine dehydrogenase catalyzes the conversion of xanthine and NAD+ to urate and NADH, providing a fundamental reducing agent necessary for redox metabolism and the production of cellular energy stores in the form of ATP.

Increases in cellular calcium lead to the activation of calcium-dependent proteases such as calpain. Calpain targets two components of the MoCo biosynthesis pathway, resulting in disruption of cellular homeostasis. Calpain irreversibly converts xanthine dehydrogenase to xanthine oxidase, creating a powerful source of reactive oxygen species that directly damage the cell. Moreover, the conversion of xanthine dehydrogenase to xanthine oxidase shunts cellular metabolism away from the production of NADH and ATP, compromising cellular energy balance. Calpain also cleaves gephyrin, resulting in loss of scaffolding function and down-regulation of inhibitory synaptic function. Calpain-cleaved gephyrin also exhibits altered MoCo synthesis function caused by the physical separation of the G and E domains.

Calpain-mediated cleavage of gephyrin at synapses creates a feedback loop in which reduced inhibitory receptor function results in increased excitatory receptor activity, increased calcium influx, and further activation of calpain.

Inflammatory cytokines such as IFNgamma and TNFalpha directly alter calcium flux in target cells and increase expression and activation of calpain. Inflammatory cytokine exposure will therefore reduce inhibitory synaptic function, increase excitatory load, alter MoCo synthesis, and drive the target cell toward reactive oxygen species production.

Inflammatory cytokines also increase the expression of GTP cyclohydrolase I, the rate limiting step in the de novo synthesis of 5,6,7,8-tetrahydrobiopterin from GTP. Inflammatory cytokine exposure will therefore shunt GTP away from MOCS1A/MOCS1AB-mediated production of cyclic pyranopterin monophosphate (cPMP), resulting in decreased MoCo synthesis.

### Seizures and the MoCo Biosynthesis Pathway

Febrile seizures are the most common type of neurologic complication in infants and preschool children. Febrile seizures occur at body temperatures over 38° C. in the absence of acute electrolyte imbalance or dehydration, in the absence of direct CNS infection, and without previous evidence of unprovoked seizures (Commission on Epidemiology and Prognosis, 1993, Epilepsia, 34:592-6). It is estimated that 1 in 25 children will experience at least one febrile seizure, and the occurrence of febrile seizure is associated with heightened susceptibility to future seizures—1 in 3 individuals with childhood febrile seizure will experience another seizure of some type within 20 years. Moreover, the risk of epilepsy among individuals experiencing a childhood febrile seizure is higher than the general population, with incidence reports ranging from 6% to 13%, rates that are more than 10 times higher than in the general population. Of note, an increased frequency of febrile seizure is associated with some vaccines in children, including measles-containing and pertussis vaccines. For example, the diptheria-tetanus-pertussis vaccine is associated with an increase of 6-9 cases of febrile seizure per 100,000 vaccinations and fever is observed in 50% of vaccinated infants. The measles-mumps-rubella (MMR) vaccine is associated with an increase of up to 16 cases of febrile seizure per 100,000 vaccinations, and the addition of varicella to the same vaccine increases the risk even further. Finally, acute seizures associated with viral, bacterial, and parasitic infections in children, whether systemic or localized to the CNS, are a primary factor in the development of epilepsy. For example, during the 2009-2010 influenza A (H1N1) pandemic, in which more than 70% of infected individuals were younger than 24 years of age, up to 6% of infections resulted in neurological complications, with over 10% of children less than 15 years of age presenting with neurological symptoms. Of these complications, seizure and abnormal EEG were the most common. Likewise, infection with enterovirus 71, a picornavirus with widespread epidemic infectivity throughout the Asia-Pacific region, is associated with neurologic complications in almost 20% of infected children. A high incidence of seizure also occurs in children infected with Plasmodium, Taenia solium and other parasites. The common factor across all of these seizure events, whether febrile or afebrile, is the production of inflammatory cytokines in the CNS. Interleukin-1beta (IL-1beta) and tumor necrosis factor alpha (TNFalpha) are powerful pyrogens that are elevated in the brain during febrile seizures, and experimental evidence directly supports a role for these factors in the initiation of seizures. Likewise, TNFalpha, interleukin-6 (IL-6), and interferon gamma (IFNgamma) are produced and/or released in the CNS during acute infection.

### Methods of Treating Neurological Inflammatory Diseases

As described herein, bypassing GTP-to-cPMP conversion by providing exogenous cPMP can stabilize MoCo synthesis and provide regulatory feedback control to drive a dysregulated system back toward homeostasis. Similarly, blocking GTP cyclohydrolase I to push GTP back into the MoCo pathway; increasing expression or activity of MOCS1A and/or MOCS1AB activity to push GTP to cPMP; or increasing expression or activity of MOCS2A, MOCS2B, and/or MOCS3 activity to push cPMP to MPT also can stabilize MoCo synthesis and provide regulatory feedback control to drive a dysregulated system back toward homeostasis. Likewise, increasing expression or activity of gephyrin to increase MoCo synthesis and to stabilize inhibitory synapses; or blocking calpain to prevent the conversion of xanthine dehydrogenase to xanthine oxidase can stabilize MoCo synthesis and provide regulatory feedback control to drive a dysregulated system back toward homeostasis. Supplementation with cPMP may be enhanced by simultaneously blocking calpain to prevent aberrant xanthine oxidase-dependent production of reactive oxygen species during an inflammatory drive and to maintain gephyrin-dependent synaptic stabilization. In addition, given the link between inflammation-induced seizures and gephyrin/GABAR, treatment with glycogen synthase kinase 3beta (GSK3beta) inhibitors may increase gephyrin activity. For example, GSK-3 Inhibitor IX (CAS 667463-62-9) or lithium chloride may suppress seizures by enhancing gephyrin function and overcome inflammation-induced shunting of gephyrin away from inhibitory synapse stabilization. Thus, methods of treating a neurological inflammatory disease are described herein.

As used herein, neurological inflammatory diseases include, without limitation, central nervous system (CNS) autoimmune disorders such as multiple sclerosis (MS), neuromyelitis optica (NMO), anti-NMDA receptor encephalitis, and autoimmune epilepsies; Alzheimer's disease; amyotrophic lateral sclerosis (ALS); schizophrenia; autism; epilepsy and other seizure disorders (e.g., febrile seizures without underlying infection); CNS infectious diseases (e.g., viral, bacterial, parasitic); MoCo deficiencies (e.g., due to genetic mutations); and other neurodegenerative diseases involving microglial and astrocytic inflammatory responses. A neurological inflammatory disease for which the methods described herein are particularly useful is neuroinflammation-induced seizures. “Treating” as used herein refers to relieving, reducing or ameliorating the symptoms of any of such neurological inflammatory diseases.

As described herein, methods of treating a neurological inflammatory disease can include administering an effective amount of cPMP to an individual. In some instances, an individual may be identified as having a neurological inflammatory disease (e.g., central nervous system (CNS) autoimmune disorders such as multiple sclerosis (MS), neuromyelitis optica (NMO), anti-NMDA receptor encephalitis, and autoimmune epilepsies; Alzheimer's disease; amyotrophic lateral sclerosis (ALS); schizophrenia; autism; epilepsy and other seizure disorders (e.g., febrile seizures without underlying infection); CNS infectious diseases (e.g., viral, bacterial, parasitic); MoCo deficiencies (e.g., due to genetic mutations); and other neurodegenerative diseases involving microglial and astrocytic inflammatory responses prior to being administered an effective amount of cPMP. In some instances, an individual may be identified as having a mutation in the MOCS1 or MOCS2 gene or the gene encoding gephyrin prior to being administered an effective amount of cPMP. cPMP can be administered on a long-term basis (e.g., when genetic mutations are present) or cPMP can be administered as an acute intervention to renormalize inhibitory synapses.

Also as described herein, methods of treating a neurological inflammatory disease can further include monitoring the individual. Simply by way of example, the amount of MPT, MoCo, MoCo—S or another intermediate or by-product of the MoCo biosynthesis pathway (e.g., levels of xanthine, hypoxanthine, uric acid, sulfite, and S-sulfocysteine) can be monitored in an individual (e.g., in urine) and can be used as biomarkers for effective cPMP dosing. In some instances, the individual's symptoms can be monitored (e.g., for improvement) or feedback from EEG can be used to monitor treatment and/or establish dosing. Depending upon the results of the monitoring step, the effective amount of cPMP can be adjusted as desired.

Typically, an effective amount of cPMP is an amount that treats (e.g., ameliorates, relieves or reduces the symptoms of) a neurological inflammatory disease without inducing any adverse effects. An effective amount of cPMP can be formulated, along with a pharmaceutically acceptable carrier, for administration to an individual. The particular formulation, will be dependent upon a variety of factors, including route of administration, dosage and dosage interval of a compound the sex, age, and weight of the individual being treated, the severity of the affliction, and the judgment of the individual's physician. As used herein, “pharmaceutically acceptable carrier” is intended to include any and all excipients, solvents, dispersion media, coatings, antibacterial and anti-fungal agents, isotonic and absorption delaying agents, and the like, compatible with administration. The use of such media and agents for pharmaceutically acceptable carriers is well known in the art. Except insofar as any conventional media or agent is incompatible with a compound, use thereof is contemplated.

Pharmaceutically acceptable carriers are well known in the art. See, for example Remington: The Science and Practice of Pharmacy, University of the Sciences in Philadelphia, Ed., 21st Edition, 2005, Lippincott Williams & Wilkins; and The Pharmacological Basis of Therapeutics, Goodman and Gilman, Eds., 12th Ed., 2001, McGraw-Hill Co. Pharmaceutically acceptable carriers are available in the art, and include those listed in various pharmacopoeias. See, for example, the U.S. Pharmacopeia (USP), Japanese Pharmacopoeia (JP), European Pharmacopoeia (EP), and British pharmacopeia (BP); the U.S. Food and Drug Administration (FDA) Center for Drug Evaluation and Research (CDER) publications (e.g., Inactive Ingredient Guide (1996)); and Ash and Ash, Eds. (2002) Handbook of Pharmaceutical Additives, Synapse Information Resources, Inc., Endicott, N.Y. The type of pharmaceutically acceptable carrier used in a particular formulation can depend on various factors, such as, for example, the physical and chemical properties of cPMP, the route of administration, and the manufacturing procedure.

A pharmaceutical composition that includes cPMP as described herein typically is formulated to be compatible with its intended route of administration. Suitable routes of administration include, for example, oral, rectal, topical, nasal, pulmonary, ocular, intestinal, and parenteral administration. Routes for parenteral administration include intravenous, intramuscular, and subcutaneous administration, as well as intraperitoneal, intra-arterial, intra-articular, intracardiac, intracisternal, intradermal, intralesional, intraocular, intrapleural, intrathecal, intrauterine, and intraventricular administration.

For intravenous injection, for example, the composition may be formulated as an aqueous solution using physiologically compatible buffers, including, for example, phosphate, histidine, or citrate for adjustment of the formulation pH, and a tonicity agent, such as, for example, sodium chloride or dextrose. For oral administration, a compound can be formulated in liquid or solid dosage forms, and also formulation as an instant release or controlled/sustained release formulations. Suitable dosage forms for oral ingestion by an individual include tablets, pills, hard and soft shell capsules, liquids, gels, syrups, slurries, suspensions, and emulsions.

Oral dosage forms can include excipients; excipients include, for example, fillers, disintegrants, binders (dry and wet), dissolution retardants, lubricants, glidants, anti-adherants, cationic exchange resins, wetting agents, antioxidants, preservatives, coloring, and flavoring agents. Specific examples of excipients include, without limitation, cellulose derivatives, citric acid, dicalcium phosphate, gelatine, magnesium carbonate, magnesium/sodium lauryl sulfate, mannitol, polyethylene glycol, polyvinyl pyrrolidone, silicates, silicium dioxide, sodium benzoate, sorbitol, starches, stearic acid or a salt thereof, sugars (e.g., dextrose, sucrose, lactose), talc, tragacanth mucilage, vegetable oils (hydrogenated), and waxes.

cPMP as described herein also can be formulated for parenteral administration (e.g., by injection). Such formulations are usually sterile and, can be provided in unit dosage forms, e.g., in ampoules, syringes, injection pens, or in multi-dose containers, the latter usually containing a preservative. The formulations may take such forms as suspensions, solutions, or emulsions in oily or aqueous vehicles, and may contain other agents, such as buffers, tonicity agents, viscosity enhancing agents, surfactants, suspending and dispersing agents, antioxidants, biocompatible polymers, chelating agents, and preservatives. Depending on the injection site, the vehicle may contain water, a synthetic or vegetable oil, and/or organic co-solvents. In certain instances, such as with a lyophilized product or a concentrate, the parenteral formulation would be reconstituted or diluted prior to administration. Polymers such as poly(lactic acid), poly(glycolic acid), or copolymers thereof, can serve as controlled or sustained release matrices, in addition to others well known in the art.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1—Effects of IFN Gamma on Neurons

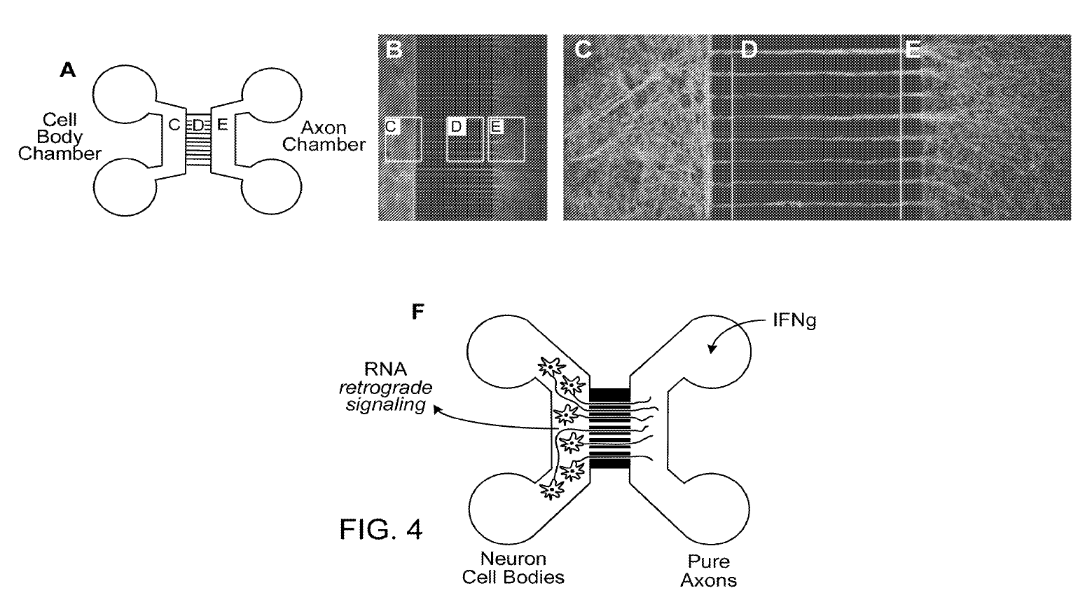

Mouse cortical neurons were cultured in a two-chamber device that separates cell bodies from axons. A schematic of the chambered device constructed in PDMS polymer is shown in FIG. 4A.

The experimental design is shown in FIG. 4F. IFN gamma was added to the pure axons in the axon chamber, and RNA was collected from the cell body chamber 72 hours later. The RNA was analyzed by microarray to identify changes in gene expression.

A low-magnification image of the regions shown in Panel A designated “C,” “D” and “E” are shown in FIG. 4B, and were stained with an antibody against neurofilament, an axon-specific protein. Higher magnification images were obtained of the cell body chamber (FIG. 4C), of the axon grooves (FIG. 4D), and of the axon chamber (FIG. 4E). DAPI staining indicated the complete absence of any cells in the axon chamber.

The results of these experiments demonstrated IFN gamma-induced dysregulation of the MoCo pathway and down-regulation of inhibitory synaptic proteins.

### Example 2—Genes Regulated by IFN Gamma

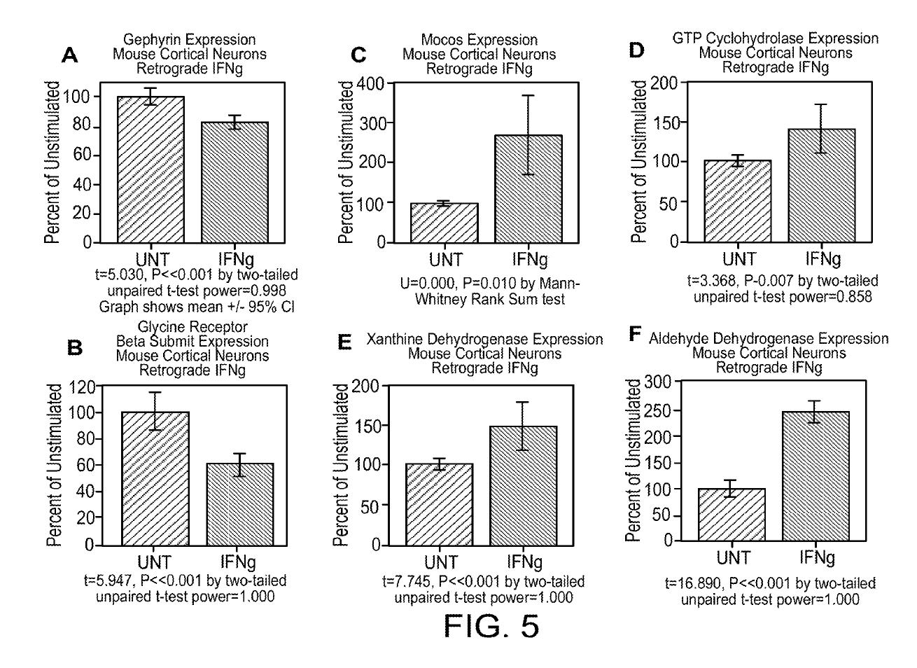

IFN gamma stimulation of the distal axons stimulated a transcriptional program in the neuron cell bodies that is marked by simultaneous down-regulation of numerous components of inhibitory synapses including, for example, gephyrin (FIG. 5A), glycine receptor beta subunit (FIG. 5B), numerous GABA receptor elements (not shown), and multiple gephyrin-binding scaffolds (not shown) as well as robust up-regulation of MOCOS (FIG. 5C). At the same time, GTP cyclohydrolase I (FIG. 5D), xanthine dehydrogenase (FIG. 5E), and aldehyde dehydrogenase (FIG. 5F) are significantly up-regulated. Taken together, these results indicate substantial changes in the MoCo pathway.

### Example 3—Pathogenic Model

While it is known that inflammatory cytokines alter neuron excitability, the underlying mechanism by which this occurs is poorly understood. Some evidence indicates that cytokines such as TNF alpha induce changes in the distribution of excitatory and inhibitory receptors on the plasma membrane of synapses, resulting in an overall alteration in excitability (Stellwagen et al., 2005, J. Neurosci., 25:3219-28). And while the impact of inflammatory cytokines on synaptic function has been widely reviewed (see, e.g., Fouregeaud and Boulanger, 2010, Eur. J. Neurosci., 32:207-17; Koller et al., 1997, Prog. Neurobiol., 52:1-26; Schafers and Sorkin, 2008, Neurosci. Lett., 437:188-93), the field stills lacks a therapeutically tractable pathogenic model for the described phenomenon.

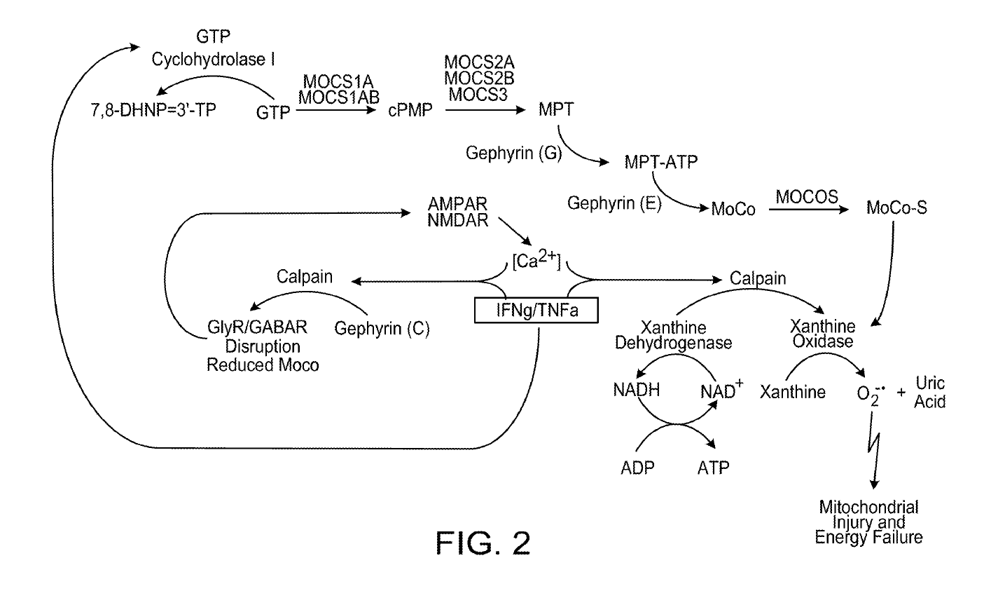

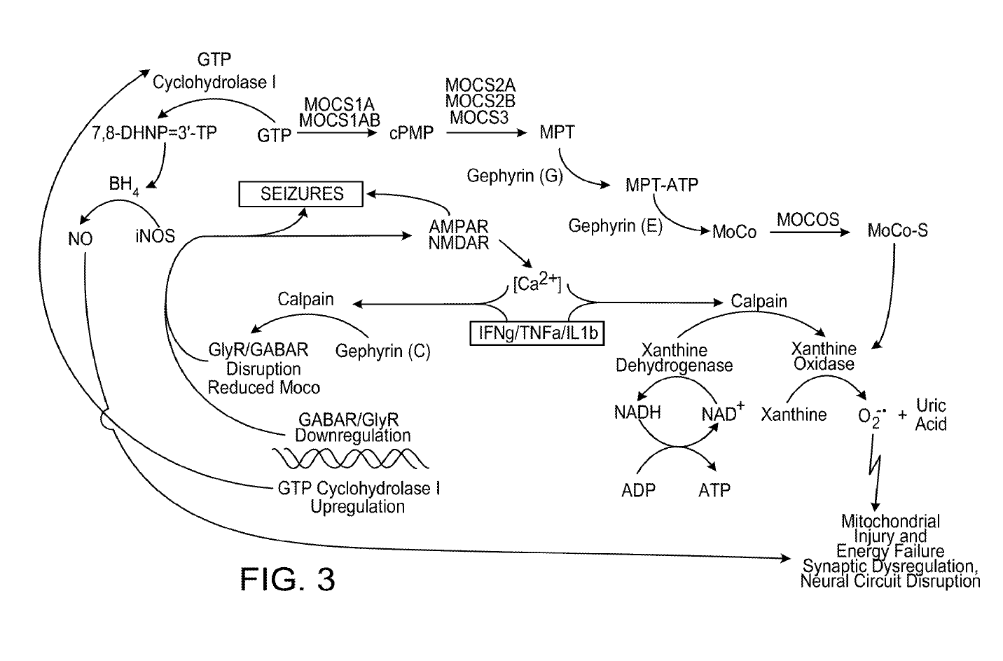

It is proposed herein that a key mechanism of hyperexcitability and seizure induction by inflammatory cytokines is the destabilization of the homeostatic molybdenum cofactor biosynthesis pathway via a reduction in gephyrin-mediated transition from MPT to MoCo, a disruption of gephyrin-mediated inhibitory neurotransmitter receptor synaptic clustering, a metabolic switch from energy production via xanthine dehydrogenase to energy failure via xanthine oxidase activity, the reversal of cPMP production from GTP to the production of 7,8-DHNP-3′-TP with concomitant amplification of nitric oxide production, the stress-dependent down-regulation of multiple components of the inhibitory neurotransmitter receptor machine, and the compensatory up-regulation of elements of the molybdenum biosynthetic apparatus. This is a novel hypothesis that places molybdenum cofactor synthesis, and particularly cPMP, at the center of a pathogenic cascade that results in severe clinical sequelae for many children. FIGS. 2 and 3 outline variations of this proposed model.

### Example 4—Expression Data

Cortical neurons were prepared from embryonic day 15 C57BL/6 mouse fetuses, following published protocols (Sauer et al., 2013, Neurobiol. Dis., 59:194-205). In preliminary experiments, after one week in vitro, the neurons were stimulated for 24 hr with IFN gamma (500 U/mL). Quadruplicate RNA samples were collected under treated and untreated conditions, and changes in gene expression were assessed using the Illumina BeadArray system. Only genes that were detected at P<0.05 on the array were considered for further analysis. Expression levels were un-normalized and the relative level of expression following IFN gamma stimulation was compared to untreated controls. Table 1 provides mean±95% confidence intervals; the appropriate statistical test was chosen based on normality and equal variance tests.

TABLE 1
IFN gamma-induced Transcriptional Changes in Mouse Cortical Neurons

| Gene | Role | Fold Change | Statistics |
|---|---|---|---|
| GABA A R α3 | inhibitory neurotransmitter receptor | 0.66 ± 0.10 | P = 0.0009 |
| | subunit | | |
| GABA A R β1 | inhibitory neurotransmitter receptor | 0.56 ± 0.09 | P = 0.0001 |
| | subunit | | |
| GABA A R β2 | inhibitory neurotransmitter receptor | 0.80 ± 0.09 | P = 0.03 |
| | subunit | | |
| GABA A R β3 | inhibitory neurotransmitter receptor | 0.75 ± 0.07 | P = 0.0009 |
| | subunit | | |
| GABA A R γ2 | inhibitory neurotransmitter receptor | 0.64 ± 0.05 | P = 0.007 |
| | subunit | | |
| GABA B R2 | inhibitory neurotransmitter receptor | 0.71 ± 0.10 | P = 0.004 |
| | subunit | | |
| Glycine R β1 | inhibitory neurotransmitter receptor | 0.59 ± 0.07 | P = 0.0001 |
| | subunit | | |
| Gephyrin | GABA and glycine receptor scaffold | 0.75 ± 0.05 | P < 0.0001 |
| | molybdenum cofactor synthesis | | |
| | catalyst | | |
| Collybistin | gephyrin clustering regulator | 0.71 ± 0.06 | P = 0.0006 |
| MOCOS | molybdenum cofactor sulfurase | 2.97 ± 0.21 | P < 0.0001 |
| Xdh | xanthine dehydrogenase; energy | 1.47 ± 0.14 | P = 0.0001 |
| | metabolism | | |
| Aldh1a1 | aldehyde dehydrogenase; metabolism | 2.46 ± 0.08 | P < 0.0001 |
| Gch1 | GTP cyclohydrolase I; biopterin | 1.59 ± 0.04 | P = 0.003 |
| | synthesis | | |
| Actb | actin; housekeeping | 1.04 ± 0.06 | P = 0.407 |
| Gapdh | glyceraldehyde 3-phosphate | 1.03 ± 0.06 | P = 0.850 |
| | dehydrogenase | | |

In order to determine the relevance of these genes to acute inflammatory events in vivo, C57BL/6 mice were infected with the Theiler's murine encephalomyelitis virus, as per standard protocols (Howe et al., 2012a, J. Neuroinflamm., 9:50; Howe et al., 2012b, Sci. Rep., 2:545; Lafrance-Corey and Howe, 2011, J. Vis. Exp., 52:2747). The hippocampus was excised at 24 hr after infection, RNA was collected, and Illumina BeadArray analysis was performed to compare gene expression levels to sham infected mice. This time point coincided with a robust inflammatory infiltrate present in the hippocampus, and it was shown that this infiltrate triggers hippocampal neural circuitry changes that result in seizures between 3 and 7 days after infection, followed by the development of epilepsy after 60 days post-infection. It was found that GTP cyclohydrolase I was up-regulated more than 4-fold in the TMEV infected mice; likewise, MOCOS was up-regulated over 3-fold, Xdh was increased by 5-fold, several aldehyde dehydrogenase isoforms were up-regulated, as was collybistin. In contrast, the delta subunit of the GABA A receptor was down-regulated by 30%, and numerous GABA receptor subunits and binding proteins were down-regulated by 10%.

### Example 5—The Impact of Inflammatory Cytokines on Molybdenum Co-Factor Biosynthesis Gene Expression and Inhibitory Neurotransmitter Receptor Function Gene Expression

Cortical and hippocampal neurons are prepared from C57BL/6 mice and are cultured under conditions that promote formation of mature synaptic networks. Cultures are exposed to TNFalpha, IL-1β, IL-6, and IFNgamma at several doses (0, 1, 3, 10, 30, 100, 300 ng/mL) and for different times (6, 12, 24, 48, 72, and 96 hr). In parallel cultures, the amount of cell death is assessed using the MTT assay, and doses that kill greater than 10% of the culture are excluded from analysis. RNA is collected using Qiagen RNeasy kits and cDNAs are generated using the Roche Transcriptor first strand cDNA synthesis kit and random hexamer primers. Probe-based real-time PCR is performed on the samples using the Roche LightCycler 480 Probes Master system, and the primer pairs and Roche Universal Probe Library hydrolysis probes defined in Table 2. Expression is normalized to Aco2 and UROD, genes that previously have been defined as suitable housekeeping factors. A multi-factor normalization scheme is used to quantify relative differences in gene expression between controls and cytokine treated samples (Anderson et al., 2004, Cancer Res., 64:5245-50).

TABLE 2
RT-PCR Analysis of Inflammatory Cytokine-Induced Changes
in MOCO Biosynthesis Pathway and Inhibitory/Excitatory Receptor Expression.

| | | SEQ | | SEQ | UPL |
|---|---|---|---|---|---|
| Gene Target | FORWARD | ID NO | REVERSE | ID NO | PROBE |
| Gephyrin var 1 (NM_145965) | tggtctcatcagttattcccatc | 1 | cgagaaatgatggagtctgga | 2 | 96 |
| Gephyrin var 2 (NM_172952) | tgatcttcatgctcagatcca | 3 | gcaaatgttgttggcaagc | 4 | 53 |
| Collybistin var 1 | tgaataaaaaggcaacctaccg | 5 | tgggagatgtcaatctctgttc | 6 | 83 |
| (NM_001033329) | | | | | |
| Collybistin var 2 | tgagaaaagcttctaaacagaaagg | 7 | gtactggccctggtttaacg | 8 | 77 |
| (NM_001290384) | | | | | |
| Mocos (NM_026779) | aagcaaagtccacacttccag | 9 | gtagtaccgggaggctgacc | 10 | 72 |
| Mocs1 (NM_020042) | ggatgtggtggacatcgtg | 11 | gaggccgatggttctcag | 12 | 11 |
| Mocs2 (NM_013826) | | 13 | tgtgagctgaagacacagca | 14 | 47 |
| Mocs3 (NM_001160330) | ggccagatgaccgtctacc | 15 | gccgtctgcacagttggt | 16 | 94 |
| [non-intron spanning] | | | | | |
| Suox (NM_173733) | tctaccatgagcatcggtgt | 17 | catcgaagacctcagagccta | 18 | 109 |
| Xdh (NM_011723) | agggattccggacctttg | 19 | gcagcagtttgggttgtttc | 20 | 69 |
| Aldh1a1 (NM_013467) | gccatcactgtgtcatctgc | 21 | catcttgaatccaccgaagg | 22 | 26 |
| Aldh1a2 (NM_009022) | catggtatcctccgcaatg | 23 | gcgcatttaaggcattgtaac | 24 | 33 |
| Aldh1a3 (NM_053080) | aacctggacaaagcactgaag | 25 | aatgcattgtagcagttgatcc | 26 | 66 |
| Aldh1a7 (NM_011921) | caagctggctgacttaatgga | 27 | gactttcccagcattcatcg | 28 | 84 |
| Aldh1b1 (NM_028270) | gaccggagaacgctgatacta | 29 | agtcgggcagtcagcatc | 30 | 45 |
| Aldh111 (NM_027406) | tccctacttcccgtctttga | 31 | acaggctctgcccgattac | 32 | 97 |
| Aldh112 (NM_153543) | cgctcgctcctctacatcat | 33 | gccaacttcagcttgtttttg | 34 | 22 |
| Aldh2 (NM_009656) | tgttcggggacgtaaaagac | 35 | tgaggatttgcatcactggt | 36 | 63 |
| Aldh3a1 (NM_007436) | tgtggagaagctcaaaaagtcac | 37 | cccatagtcatgggactgct | 38 | 60 |
| Aldh3a2 (NM_007437) | gctgaagcagttcaacaaagg | 39 | aggagaggcaacaaggaagtc | 40 | 89 |
| Aldh3b1 (NM_026316) | ctggatgaagccatcgagtt | 41 | ccaacacctgtttgataacctg | 42 | 7 |
| Aldh3b2 (NM_001177438) | aagagttgctgcacatctgg | 43 | gtgcagtgtggcctcaga | 44 | 84 |
| Aldh4a1 (NM_175438) | accggttccgtaccttcc | 45 | agctgtgcacaaaatggaaat | 46 | 19 |
| Aldh5a1 (NM_172532) | aacagctggaaaggggtctc | 47 | cattaagtcgtaccatttacggagt | 55 | |
| Aldh6a1 (NM_134042) | ggtaaatgccacatggtatcc | 49 | tgattcaacaaattttccatcaat | 88 | |
| Aldh7a1 (NM_138600) | tgtccttcactgggagcact | 51 | gtttcctccaagctccaaca | 52 | 10 |
| Aldh8a1 (NM_178713) | ggcaagaatcctgctatcatct | 53 | actggtacaaaggcaaatctcc | 54 | 25 |
| Aldh9a1 (NM_019993) | ctggcagtgcctggaatact | 55 | cctcctgggagctgaatgt | 56 | 71 |
| Aldh16a1 (NM_145954) | ctcttgggccaccatgtc | 57 | ctggccaggttttctcctg | 58 | 5 |
| Aldh18a1 (NM_019698) | gatgtcatcgtcacagagaacg | 59 | aagacacaggcgctgtcc | 60 | 67 |
| GTP Cyclohydrolase 1 Gch1 | gcctcaccaaacagattgc | 61 | cacgcctcgcattaccat | 62 | 40 |
| (NM_008102) | | | | | |
| Nitric Oxide Synthase 1 | ggcgttcgtgattactgtga | 63 | tcttcctcatgtccaaatcca | 64 | 69 |
| (neuronal) Nos1 | | | | | |
| (NM_008712) | | | | | |
| Nitric Oxide Synthase 2 | ctttgccacggacgagac | 65 | tcattgtactctgagggctgac | 66 | 13 |
| (inducible) Nos2 | | | | | |
| (NM_010927) | | | | | |
| Glycine Receptor Glra1 | caacacaaggaactgcttcg | 67 | gatttagcatggggctcttg | 68 | 69 |
| (NM_001290821) | | | | | |
| Glycine Receptor Glra2 | cataaggagttccttcgtctc | 69 | cgactttcacgagtaacatcttctt | 70 | 34 |
| (NM_183427) | | | | | |
| Glycine Receptor Glra3 | gggaagccgcactgttact | 71 | gagatcgcgcactgtttgt | 72 | 49 |
| (NM_080438) | | | | | |
| Glycine Receptor Glrb1 | ctgatgctagtgctgccaga | 73 | gtgcactctgaggccaaact | 74 | 105 |
| (NM_010298) | | | | | |
| GABA A Receptor Gabra1 | gcccactaaaattcggaagc | 75 | cttctgctacaaccactgaacg | 76 | 93 |
| (NM_010250) | | | | | |
| GABA A Receptor alpha | acaaaaagaggatgggcttg | 77 | tcatgacggagcctttctct | 78 | 103 |
| Gabra2 (NM_008066) | | | | | |
| GABA A Receptor alpha | cttgggaaggcaagaaggta | 79 | tggagctgctggtgttttct | 80 | 80 |
| Gabra3 (NM_008067) | | | | | |
| GABA A Receptor alpha | aaagcctcccccagaagtt | 81 | catgttcaaattggcatgtgt | 82 | 68 |
| Gabra4 (NM_010251) | | | | | |
| GABA A Receptor alpha | caagtgtctttctggattaataaggag | 83 | gtcatggttaaaacagtggtgatt | 84 | 95 |
| Gabra6 (NM_001099641) | | | | | |
| GABA A Receptor beta | ccctctggatgagcaaaact | 85 | aattcgatgtcatccgtggta | 86 | 80 |
| Gabrb1 (NM_008069) | | | | | |
| GABA A Receptor beta | gggtctccttttggattaactatga | 87 | ggtcattgttaggacagttgtaattc | 88 | 84 |
| Gabrb2 (NM_008070) | | | | | |
| GABA A Receptor beta | ctccattgtagagcaccgtct | 89 | tcaatgaaagtcgaggataggc | 90 | 80 |
| Gabrb3 (NM_008071) | | | | | |
| GABA A Receptor delta | cggagctgatgaacttcaaat | 91 | atgtagacgccccggttc | 92 | 11 |
| Gabrd (NM_008072) | | | | | |
| GABA A Receptor gamma 2 | acagaaaatgacgctgtgga | 93 | catctgacttttggcttgtgaa | 94 | 68 |
| Gabrg2 (NM_008073) | | | | | |
| GABA A Receptor gamma 3 | atgcgacaccagcaagaac | 95 | caatggtgctgagtgtggtc | 96 | 9 |
| Gabrg3 (NM_008074) | | | | | |
| GABA A Receptor pi Gabrp | atttcaccctggtcaccgta | 97 | gctcaaattgcaaaaccaatc | 98 | 18 |
| (NM_146017) | | | | | |
| GABA A Receptor rho Gabrr1 | tgcctgctagagtcccctta | 99 | ccgtgatgatggtggacat | 100 | 9 |
| (NM_008075) | | | | | |
| GABA B Receptor 1 Gabbr1 | gacattgatgtctccattctgc | 101 | gcagccctttgtaaccataga | 102 | 78 |
| (NM_019439) | | | | | |
| GABA B Receptor 2 Gabbr2 | gaacatggcagcgaaagtct | 103 | ctggtacttgctgccaaaca | 104 | 91 |
| (NM_001081141) | | | | | |
| Glutamate Receptor AMPA 1 | agggatcgacatccagagag | 105 | tgcacatttcctgtcaaacc | 106 | 62 |
| Gria1 (NM_008165) | | | | | |
| Glutamate Receptor AMPA 2 | ggggaggtgattccaagg | 107 | cccccgacaaggatgtaga | 108 | 67 |
| Gria2 (NM_013540) | | | | | |
| Glutamate Receptor AMPA 3 | agccgtgtgatacgatgaaa | 109 | caaggtttacaggcgttcct | 110 | 31 |
| Gria3 (NM_016886) | | | | | |
| Glutamate Receptor AMPA 4 | ctgccaacagttttgctgtg | 111 | aaatggcaaacacccctcta | 112 | 48 |
| Gria4 (NM_019691) | | | | | |
| Glutamate Receptor Delta 1 | agacttttggcaaagacatgc | 113 | actgccattcaagcccttc | 114 | 95 |
| Grid1 (NM_008166) | | | | | |
| Glutamate Receptor Delta 2 | ccctaccgtgatgtcttttca | 115 | agaatgtccatgtcgccact | 116 | 1 |
| Grid2 (NM_008167) | | | | | |
| Glutamate Receptor Kainate | tctggtttggcgttggag | 117 | tcctccaactattctggtcgat | 118 | 105 |
| 1 Grik1 (NM_146072) | | | | | |
| Glutamate Receptor Kainate | agtgccaccataccatccag | 119 | gctggcacttcagagacattc | 120 | 31 |
| 2 Grik2 (NM_010349) | | | | | |
| Glutamate Receptor Kainate | cacttcatcttcaccactctgg | 121 | actcccgagtagcggtagg | 122 | 85 |
| 3 Grik3 (NM_001081097) | | | | | |
| Glutamate Receptor Kainate | gccattgagtatggcacgat | 123 | tggtaacgggaattttggaa | 124 | 67 |
| 4 Grik4 (NM_175481) | | | | | |
| Glutamate Receptor Kainate | cccctcagctagcctcatct | 125 | gcctcgcaccagttcttcta | 126 | 40 |
| 5 Grik5 (NM_008168) | | | | | |
| Glutamate Receptor NMDA 1 | catttagggctatcacctcca | 127 | cactgtgtctttttggttttgc | 128 | 78 |
| Grin1 (NM_008169) | | | | | |
| Glutamate Receptor NMDA 2A | attcaaccagaggggcgta | 129 | ttcaagacagctgcgtcatag | 130 | 48 |
| Grin2a (NM_008170) | | | | | |
| Glutamate Receptor NMDA 2B | gggttacaaccggtgccta | 131 | ctttgccgatggtgaaagat | 132 | 53 |
| Grin2b (NM_008171) | | | | | |
| Glutamate Receptor NMDA 2C | gaagcgggccatagacct | 133 | tggcagatccctgagagc | 134 | 94 |
| Grin2c (NM_010350) | | | | | |
| Glutamate Receptor NMDA 2D | tgcgatacaaccagccaag | 135 | agatgaaggcgtccagtttc | 136 | 25 |
| Grin2d (NM_008172) | | | | | |
| Glutamate Receptor NMDA 3A | cacgaatcaaaaacaaatccaa | 137 | tgtgtttaatgctctgtgaaacc | 138 | 71 |
| Grin3A (NM_001276355) | | | | | |
| Glutamate Receptor NMDA 3B | cgtcctacggagggaggt | 139 | ccagggacaccagcacat | 140 | 80 |
| Grin3b (NM_130455) | | | | | |
| Glutamate Receptor NMDA | ctcaaggaggctacccacag | 141 | tagttcccatgctgaggtga | 142 | 77 |
| AP1 Grina (NM_023168) | | | | | |
| PSD-95 D1g4 (NM_007864) | tctgtgcgagaggtagcaga | 143 | cggatgaagatggcgatag | 144 | 102 |

### Example 6—The Impact of Inflammatory Cytokines on Molybdenum Co-Factor Biosynthesis and Inhibitory Neurotransmitter Receptor Function Protein Expression

Similar cultures and treatment conditions as described in Example 5 are used to generate protein lysates for analysis of expression of GABA receptor subunits, glycine receptor subunits, gephyrin, GTP cyclohydrolase I, and MoCoS. Neurons grown in glass multi-well chambered slides are used for the analysis of expression of these targets by immunofluorescence microscopy. For IF, cells are stimulated for 24, 48, 72, or 96 hrs at 100 ng/mL (or at a dose defined in Example 5 as optimal for gene induction) prior to fixation and immunostaining. Table 3 lists the relevant antibodies that are employed.

TABLE 3
Antibodies

| Target | Source |
|---|---|
| GABA A R α1 | Synaptic Sys #224203, 1:500 (WB), rabbit |
| | Alomone #AGA-001, 1:400 (IF), rabbit |
| GABA A R α2 | Synaptic Sys #224102, 1:500 (WB), rabbit |
| | Alomone #AGA-002, 1:200 (IF), rabbit |
| GABA A R α3 | Synaptic Sys #224302, 1:500 (WB), rabbit |
| | Alomone #AGA-003, 1:200 (IF), rabbit |
| GABA A R β2/3 | Millipore #MAB341, 1:50 (WB), 1:25 (IF), mouse |
| GABA A R γ2 | Alomone #AGA-005, 1:200 (WB), 1:100 (IF), rabbit |
| GABA B R2 | Synaptic Sys #322205, 1:1000 (WB), 1:500 (IF), |
| | guinea pig |
| Glycine R β | Alomone #AGR-014, 1:200 (WB), rabbit |
| | Synaptic Sys #146211, 1:500 (IF), mouse |
| Gephyrin | Synaptic Sys #147004, 1:1000 (WB), guinea pig |
| | Synaptic Sys #147021, 1:250 (IF), mouse |
| Collybistin | Synaptic Sys #261003, 1:1000 (WB), 1:500 (IF), |
| | rabbit |
| GTP | Sigma #SAB4200046-200UL, 1:1000 (WB) |
| Cyclohydrolase I | |
| MoCo-S | Santa Cruz #SC-85066, 1:200 (WB), rabbit |
| | Pierce #PA5-30662, 1:500 (WB), rabbit |

### Example 7—The Impact of Inflammatory Cytokines on Spontaneous and Evoked Synaptic Activity

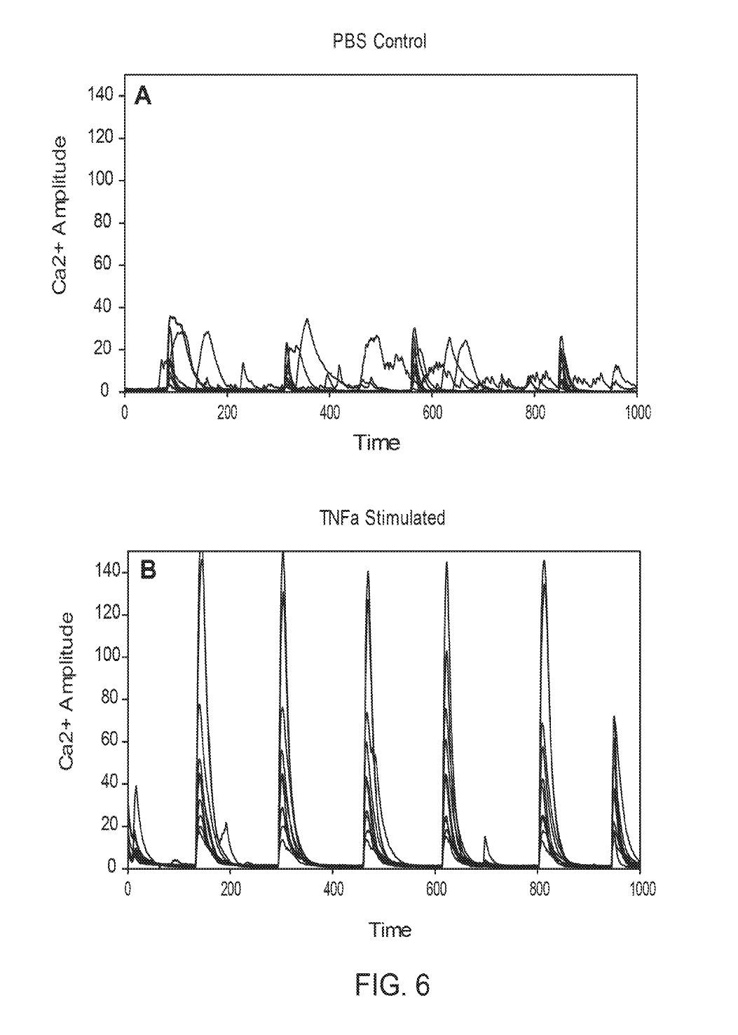

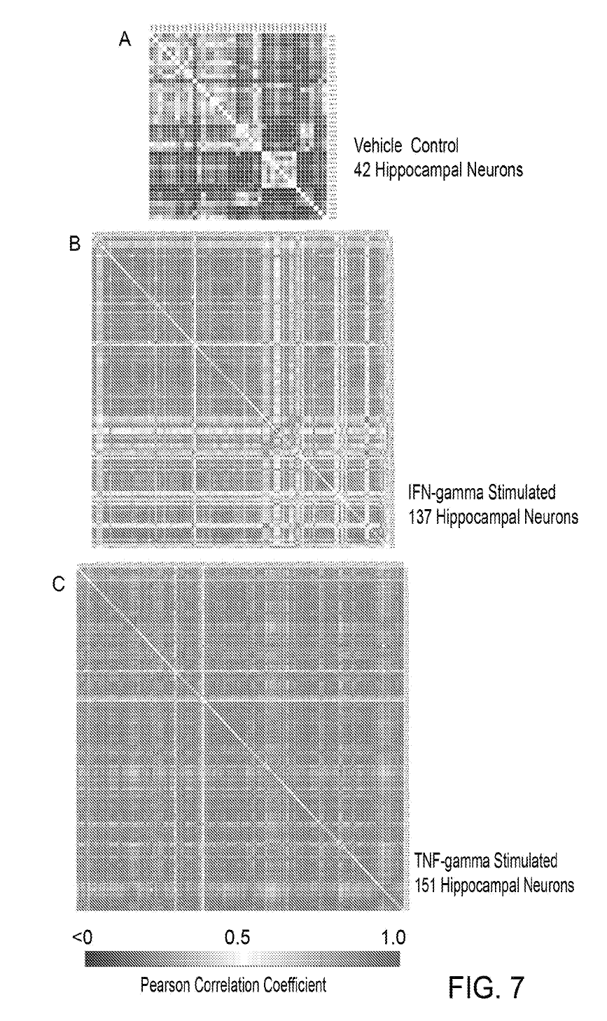

Neurons are cultured in glass imaging chambers under conditions that promote formation of mature synaptic networks. Cells are infected with an AAV1.Syn.GCaMP6f calcium reporter that provides fast optical tracking of intracellular calcium levels (Akerboom et al., 2012, J. Neurosci., 32:13819040; Chen et al., 2013, Nature, 499:295-300). Calcium levels are monitored in real-time using a Zeiss 5-Live confocal microscope equipped with environmental chamber. Following collection of baseline spontaneous activity levels at low magnification, inflammatory cytokine is added at the optimal cytokine concentration determined above, and cells are followed for up to 60 minutes. Images are post-processed in Image J to measure calcium transient amplitudes and frequencies within defined cells. In some experiments, an Olympus multi-photon microscope is used at high magnification to track activity in individual synapses. In addition to spontaneous activity, calcium flux elicited by addition of glutamate to cultures that have been pretreated with inflammatory cytokines for different times (0, 1, 3, 6, 12, 24, 48, 72, or 96 hr) prior to stimulation also is measured. See FIGS. 6 and 7.

### Example 8—The Effect of cPMP Supplementation on Synaptic Changes Induced by Inflammatory Cytokines

Hippocampal and cortical neurons are treated with inflammatory cytokines at the optimized dose and time identified above in the presence of different concentrations of cPMP. Extrapolating from the field of purinergic signaling, concentrations ranging from nanomolar to millimolar (1, 3, 10, 30, 100, 300 nM; 1, 3, 10, 30, 100, 300 μM; 1, 3 mM) are tested. In preliminary experiments, the survival of naive neurons treated with cPMP for different times (1, 3, 6, 12, 24, 48, 72, or 96 hr) is assessed by MTT or LDH assay, and doses that kill more than 10% of cells are excluded from further analysis. In some instances, the cPMP is encapsulated in liposomes (for example, lipofectin or lipofectamine) (Hughes et al., 2010, Methods Mol. Biol., 605:445-59). After optimizing cPMP delivery, neurons are stimulated with inflammatory cytokines in the presence or absence of cPMP under conditions that alter spontaneous and/or evoked calcium flux. If cPMP treatment reverses the effect of inflammatory cytokines on dynamic synaptic activity, the effect of cPMP on expression and localization of the protein targets explored in Example 6 also is examined, and the effect of cPMP on the expression of genes measured in Example 5 is tested.

### Example 9—MoCo Pathway and Neurotransmitter Receptor Changes Induced by Acute Virus Infection of the Brain

Young (4 week old) mice were infected with the Theiler's murine encephalomyelitis virus for 24 hr to model acute childhood brain infection. Illumina microarray was employed to assess transcriptional changes. Table 4 shows maximal up-regulation or down-regulation of relevant genes during the first 24 hr of infection.

TABLE 4
Maximal Up-regulation or Down-regulation of Relevant Genes

| | Fold- | | | Fold- | |
|---|---|---|---|---|---|
| Gene | change | Function | Gene | change | Function |
| MoCos | 3.0 | molybdenum cofactor | Gphn | −1.4 | gephyrin (Mo |
| | | biosynthesis | | | pathway and |
| | | | | | inhibitory synapse) |
| Xdh | 4.9 | xanthine | Glra2 | −1.4 | glycine receptor |
| | | dehydrogenase/oxidase | | | (inhibitory) |
| Gch1 | 4.3 | GTP cyclohydrolase 1 | Gabrd | −1.6 | GABA receptor |
| | | | | | (inhibitory) |
| Aldh1b1 | 2.5 | aldehyde dehydrogenase 1 | Gabrb1 | −1.4 | GABA receptor |
| | | | | | (inhibitory) |
| Nos3 | 1.3 | nitric oxide synthase 3 | Gabrg1 | −1.3 | GABA receptor |
| | | | | | (inhibitory) |
| Gria2 | 1.3 | AMPA receptor | Gabbr2 | −1.3 | GABA receptor |
| | | (excitatory) | | | (inhibitory) |
| Grin2c | 1.2 | NMDA receptor | Gabbr3 | −1.3 | GABA receptor |
| | | (excitatory) | | | (inhibitory) |
| Dlg5 | 1.3 | disks large homolog | Gabbr1 | −1.3 | GABA receptor |
| | | (excitatory) | | | (inhibitory) |
| Capn2 | 1.5 | large calpain subunit | Gabra1 | −1.3 | GABA receptor |
| | | | | | (inhibitory) |
| Capn5 | 1.3 | large calpain subunit | Gad1 | −1.3 | glutamate |
| | | | | | decarboxylase |
| | | | | | (inhibitory) |
| Capns1 | 1.3 | small calpain subunit | | | |
| Gpx1 | 1.5 | glutathione peroxidase 1 | | | |
| Akr1b8 | 1.8 | aldo/keto reductase 1 | | | |
| G6pdx | 1.3 | glucose-6-P | | | |
| | | dehydrogenase | | | |

These measurements indicate that acute infection of the brain, consistent with elevated TNF alpha (6-fold increase at 24 hr) and IL1 beta (10-fold increase at 24 hr) in this model system, induces increased synthesis of MoCo pathway-related factors, increased production of oxidative stress factors, up-regulated calpain production, and increased expression of excitatory neurotransmitter receptors. Simultaneously, acute infection triggers down-regulated expression of gephyrin and a host of GABAergic receptors, resulting in suppression of synaptic inhibition.

### Example 10—MoCo Pathway and Neurotransmitter Receptor Changes in Human Neurons Induced by Inflammatory Cytokines

Neurons were induced from human neural stem cells and grown under conditions that foster mixed excitatory and inhibitory neuron phenotypes. These cells were then stimulated with TNF alpha, IL1 beta, or IFN gamma for 24 hrs, and transcriptional changes were assessed by microarray. Responses were variable between cytokines but, in general, the inflammatory stimuli induced changes that are summarized in Table 5.

TABLE 5
Inflammatory Stimuli Induced Genes

| | Fold- | | | Fold- | |
|---|---|---|---|---|---|
| Gene | change | Function | Gene | change | Function |
| MoCos | 2.0 | molybdenum cofactor | Gphn | −1.2 | gephyrin (Mo |
| | | biosynthesis | | | pathway and |
| | | | | | inhibitory synapse) |
| Gch1 | 3.7 | GTP cyclohydrolase 1 | Glra2 | −1.7 | glycine receptor |
| | | | | | (inhibitory) |
| Aldh1 | 1.5 | aldehyde | Glrb | −1.7 | glycine receptor |
| | | dehydrogenase 1 | | | (inhibitory) |
| Aldh2 | 1.4 | aldehyde | Gabrd | −1.6 | GABA receptor |
| | | dehydrogenase 2 | | | (inhibitory) |
| Nos3 | 1.6 | nitric oxide synthase 3 | Gabbr2 | −5.0 | GABA receptor |
| | | | | | (inhibitory) |
| Gria3 | 1.4 | AMPA receptor | Gabbr3 | −2.5 | GABA receptor |
| | | (excitatory) | | | (inhibitory) |
| Grina | 1.4 | NMDA receptor | Gad1 | −1.8 | glutamate |
| | | (excitatory) | | | decarboxylase |
| | | | | | (inhibitory) |
| Grin3a | 2.3 | NMDA receptor | | | |
| | | (excitatory) | | | |
| Grik2 | 1.5 | | | | |
| Grm5 | 3.9 | metabotropic | | | |
| | | glutamate receptor | | | |
| | | (excitatory) | | | |
| Dlg3 | 1.2 | disks large homolog | | | |
| | | (excitatory) | | | |
| Capn2 | 1.5 | large calpain subunit | | | |
| Capn5 | 5.3 | large calpain subunit | | | |
| Capns1 | 1.2 | small calpain subunit | | | |

### Example 11—MoCo Pathway and Neurotransmitter Receptor Changes in Human Neurons Induced by Inflammatory Cytokines

Neurons were cultured from neonatal mice and stimulated with TNF alpha or IFN gamma for 24 hr. Transcriptional changes were assessed by quantitative RT-PCR.

TABLE 6
Transcriptional Changes

| | Fold- | | | Fold- | |
|---|---|---|---|---|---|
| Gene | change | Function | Gene | change | Function |
| Gria1 | 1.2 | AMPA receptor | Gphn | −2.1 | gephyrin (Mo |
| | | (excitatory) | | | pathway and |
| | | | | | inhibitory synapse) |
| Gria2 | 1.7 | AMPA receptor | Glra2 | −1.6 | glycine receptor |
| | | (excitatory) | | | (inhibitory) |
| Gria3 | 4.1 | AMPA receptor | Glra3 | −2.3 | glycine receptor |
| | | (excitatory) | | | (inhibitory) |
| Grid1 | 1.3 | glutamate receptor | Glrb1 | −4.5 | glycine receptor |
| | | (excitatory) | | | (inhibitory) |
| Grid2 | 2.4 | glutamate receptor | Gabra1 | −1.2 | GABA receptor |
| | | (excitatory) | | | (inhibitory) |
| Grik3 | 6.2 | kainate receptor | Gabra2 | −2.3 | GABA receptor |
| | | (excitatory) | | | (inhibitory) |
| Grik4 | 3.0 | kainate receptor | Gabra4 | −2.9 | GABA receptor |
| | | (excitatory) | | | (inhibitory) |
| Grik5 | 1.8 | kainate receptor | Gabrb1 | −1.4 | GABA receptor |
| | | (excitatory) | | | (inhibitory) |
| Grin2b | 2.6 | NMDA receptor | Gabrd | −1.6 | GABA receptor |
| | | (excitatory) | | | (inhibitory) |
| Grin2d | 1.9 | NMDA receptor | Gabrg2 | −4.8 | GABA receptor |
| | | (excitatory) | | | (inhibitory) |
| Grin3a | 2.7 | NMDA receptor | | | |
| | | (excitatory) | | | |
| Grina | 2.6 | NMDA receptor | | | |
| | | (excitatory) | | | |

### Example 12—Inflammatory Cytokines Induce Network Synchronization that Phenocopies Loss of Inhibition

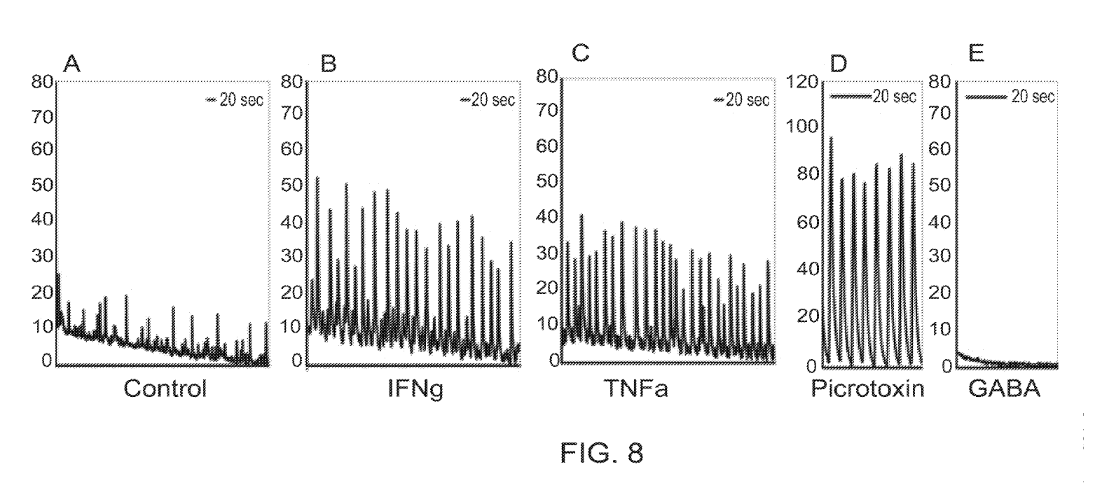

Neurons were cultured from neonatal mice and stimulated with TNF alpha (100 ng/mL) or IFN gamma (500 U/mL) for 24 hr. Following transduction with an AAV-encoded GCaMPf reporter, fast calcium transients were imaged in the cells. Regions of interest outlining individual neurons were defined in each frame of movies collected over several minutes, and the fluorescence intensity of each cell was graphed through time to reveal patterns in the population response. FIG. 8 shows the averaged calcium response traces calculated for dozens of cytokine-stimulated neurons in each experiment and are representative of more than 4 separate experiments and more than 4 separate cell preps within each experiment.

FIG. 8A shows the basal level of calcium activity in the neuron cultures. Non-synchronized calcium responses occur in the control cultures, resulting in an overall low level of synaptic activity in the network. FIGS. 8B and 8C show the stimulation of calcium activity in neurons following treatment with IFN gamma or TNF alpha, respectively. FIGS. 8B and 8C show that treatment with IFN gamma or TNF alpha results in network bursting and highly synchronized synaptic activity in which many cells in the culture flux calcium at the same time.

FIG. 8D shows that treatment of control cultures with 2.4 μM picrotoxin, a small molecule inhibitor of inhibitory GABAergic channels, induces network synchrony and bursting that phenocopies the response observed in cytokine-stimulated cultures (compare with FIGS. 8B and 8C). FIG. 8E shows that addition of 27 μM GABA to control cultures completely suppresses synaptic activity, consistent with enhanced inhibition.

These findings indicate that TNF alpha and IFN gamma induce the suppression of inhibitory neurotransmission in the neuronal network, resulting in synchronous bursting behavior. Given the transcriptional profiles measured in cytokine-stimulated neurons, this network behavior is consistent with a reduction in inhibitory neurotransmitter receptors linked to reduced gephyrin expression and alteration of the MoCo synthesis pathway.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. A method of treating a neurological inflammatory disease in an individual, comprising:
administering an effective amount of cPMP to the individual,
thereby treating the individual.

2. The method of claim 1, wherein the neurological inflammatory disease is selected from the group consisting of central nervous system (CNS) autoimmune disorders such as multiple sclerosis (MS); neuromyelitis optica (NMO); anti-NMDA receptor encephalitis; autoimmune epilepsies; Alzheimer's disease; amyotrophic lateral sclerosis (ALS); schizophrenia; autism; epilepsy; febrile seizures without underlying infection; CNS viral, bacterial, or parasitic infectious diseases; MoCo deficiencies; and other neurodegenerative diseases involving microglial and astrocytic inflammatory responses.

3. The method of claim 1, wherein the administering step is selected from the group consisting of orally, topically, and parenterally.

4. The method of claim 1, further comprising identifying an individual having a neurological inflammatory disease.

5. The method of claim 1, further comprising identifying an individual having a mutation in a gene selected from the group consisting of gephyrin, MOCS1, and MOCS2.

6. The method of claim 1, further comprising identifying an individual having ALS.

7. The method of claim 1, further comprising identifying an individual having epilepsy or another seizure disorder.

8. The method of claim 1, further comprising identifying an individual having autism or schizophrenia.

9. The method of claim 1, further comprising monitoring the individual for the amount of MPT, MoCo, MoCo—S or another intermediate or by-product of the MoCo biosynthesis pathway.

10. A method of treating ALS, epilepsy or another seizure disorder in an individual, comprising:
administering an effective amount of cPMP to the individual,
thereby treating the individual.

11. The method of claim 10, wherein the administering step is selected from the group consisting of orally, topically, and parenterally.

12. The method of claim 10, further comprising identifying an individual having ALS, epilepsy or another seizure disorder.

13. The method of claim 10, further comprising monitoring the individual for the amount of MPT, MoCo, MoCo—S or another intermediate or by-product of the MoCo biosynthesis pathway.

14. A method of treating neuroinflammation-induced seizures in an individual, comprising:
administering an effective amount of cPMP to the individual,
thereby treating the individual.
